(19) United States
(12) Patent Application Publication
(10) Pub. No.: US 2024/0009437 A1
(43) Pub. Date: Jan. 11, 2024

(54) MEANS AND METHODS FOR IMPLANTING CELLS SECRETING A THERAPEUTIC COMPOUND

(71) Applicant: Roche Diabetes Care, Inc., Indianapolis, IN (US)

(72) Inventors: Florian Diehr, Lampertheim (DE); Alexander Licht, Weinheim (DE); Arnulf Staib, Heppenheim (DE)

(21) Appl. No.: 18/452,096

(22) Filed: Aug. 18, 2023

Related U.S. Application Data
(63) Continuation of application No. PCT/EP2022/053904, filed on Feb. 17, 2022.

(30) Foreign Application Priority Data
Feb. 19, 2021 (EP) 21 158 242.4
Mar. 10, 2021 (EP) 21 161 809.5

(51) Int. Cl. A61M 39/02; A61L 27/38; A61L 27/54; A61L 27/48; A61K 38/28

(52) U.S. Cl. CPC A61M 39/02; A61L 27/38; A61L 27/54; A61L 27/48; A61K 38/28; A61M 2207/00; A61L 2300/43

(57) ABSTRACT

A device for implantation into a mammalian host has a dialysis tube with a lumen and a dialysis membrane enclosing the lumen. The dialysis tube has an outer diameter of 20 μm to 600 μm. The dialysis membrane has a molecular weight cutoff of from 25 kDa to 150 kDa and has a material composition of at least one of the following: polyethersulfone, polyarylethersulfone, polyethylene, polytetrafluoroethylene, polyvinylidene fluoride, and polypropylene. A therapeutic composition is disposed within the lumen and has cells that secrete a therapeutic compound. A matrix material embeds the cells. The matrix material is one or more of alginate, collagen, fibrin, extracellular matrix material, synthetic hydrogel, and acrylate. Also disclosed are a method of producing a device for implantation, a port system, and a cell secreting a therapeutic compound for use in treating disease by implantation of the cells in a device for implantation.

### RELATED APPLICATIONS

This application is a continuation of International Application Serial No. PCT/EP2022/053904, filed Feb. 17, 2022, which claims priority to EP 21 161 809.5, filed Mar. 10, 2021, and EP 21 158 242.4, filed Feb. 19, 2021, the entire disclosures of all of which are hereby incorporated herein by reference.

### BACKGROUND

This disclosure relates to a device for implantation into a mammalian host, the device having a dialysis tube with a lumen and a dialysis membrane enclosing the lumen.

Therapeutic proteins are used at an increasing rate for treatment of chronic disease, e.g., in treatment of neurodegenerative diseases, hemophilia, cancer, anemia, diabetes, autoimmune disease, and the like. Typically, the therapeutic polypeptide is administered by repeated injection or infusion, causing variations in bioavailability of the compound; also the need for repeated administration may cause decrease in compliance. Thus, devices permitting production of therapeutic polypeptides in the body of a patient were devised. Important requirements for such devices are safety for the patient, production of the therapeutic polypeptide over extended periods, and ability to replace in case production of the therapeutic compound decreases below the desirable level. Moreover, it is deemed desirable to be able to remove the cells producing the therapeutic polypeptide from the body of the patient, e.g., in case an allergic reaction to these cells is developed.

Basic requirements for implantation of cells into a patient are described, e.g., in Fotino et al. (2015), Pharmacol Res, 98, 76-85; also, a variety of devices has been proposed by several companies, e.g., ViaCyte, Semma Therapeutics, Beat 02 Technologies, and Defymed®.

AU 2020 210244 A1 and CA 2190628 C teach devices for implantation of insulin producing cells, having large pores with a diameter in the range of 0.4 μm, and an inner diameter of more than 1.5 mm. WO 1997/013474 A1 discloses a flat sheet type device for implantation into a mammalian host, in which cells producing insulin are embedded in alginate and the admixture is further covered with another alginate layer. WO 2014/202199 A1 also teaches a device for implantation comprising a dialysis tube with pores in the range of from 40 to 290 μm.

Nonetheless, the solutions provided by the prior art were not optimal under at least one of the aforesaid desirable aspects, in particular productivity over extended periods of time and having an option to replace cells in case production of the therapeutic compound decreases below the desirable level. Thus, there is a need for improved means and methods for implanting cells producing therapeutic compounds into a mammalian host, avoiding at least in part the drawbacks of the prior art.

### SUMMARY

This disclosure relates to a device for implantation into a mammalian host, the device comprising:
a) a dialysis tube with a lumen and a dialysis membrane enclosing said lumen, wherein
   (i) the dialysis tube has an outer diameter of from 20 μm to 600 μm; and
   (ii) the dialysis membrane has a molecular weight cutoff of from 25 kDa to 150 kDa and comprises at least one material selected from the group consisting of polyethersulfone, polyarylethersulfone, polyethylene, polytetrafluoroethylene, polyvinylidene fluoride, and polypropylene; and
b) a therapeutic composition disposed within the lumen of the dialysis tube, wherein said therapeutic composition comprises a plurality of cells secreting a therapeutic compound and a matrix material embedding said cells secreting a therapeutic compound, wherein the matrix material is selected from alginate, collagen, fibrin, extracellular matrix material, synthetic hydrogel, and acrylate, in an embodiment the matrix material is alginate.

In general, terms used herein are to be given their ordinary and customary meaning to a person of ordinary skill in the art and, unless indicated otherwise, are not to be limited to a special or customized meaning. As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more,” i.e., are equivalent to “comprising at least one.”

Further, as used in the following, the terms “preferably,” “in an embodiment,” “in a further embodiment,” “particularly,” “more particularly,” “specifically,” “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “dialysis tube,” “dialysis membrane,” and “port,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

As used herein, the term “standard conditions,” if not otherwise noted, relates to IUPAC standard ambient temperature and pressure (SATP) conditions, i.e., preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e., potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of this disclosure. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The term “device for implantation,” as used herein, relates to each and every device having the features as specified herein and being adapted for implantation into a mammalian host. Adaptation for implantation comprises that the device for implantation has an overall size and structure making implantation into the body of a mammalian host possible. As the skilled person understands, the specific size and structure requirement for the device for implantation may vary and depend, among other factors, on the nature of the mammalian host, e.g., its species, size, and/or age, as well as on the intended location of implantation. In an embodiment, the device for implantation is implanted in a body cavity of the mammalian host, in an embodiment a natural or an artificially created body cavity, in a further embodiment a natural body cavity. Natural body cavities are in particular the abdomen, the peritoneum, the circulation system, the oropharynx cavity, the digestive tract, the lung, the uterus, and the vagina. Artificial body cavities may be created, e.g., in a subcutaneous tissue, a fat tissue, a muscle, the cranium, the pleura, or a breast of the mammalian host.

In an embodiment, adaptation for implantation further comprises not being detrimental to the mammalian host, in particular in cases where the device for implantation is maintained in an implanted state for an extended periods of time, such as at least 1 week, in an embodiment at least 2 weeks, in a further embodiment at least 1 month, in a further embodiment at least 6 months, in a further embodiment at least one year. Thus, in an embodiment, the device for implantation comprises biocompatible materials, in a further embodiment consists of biocompatible surface materials, in a further embodiment consists of biocompatible materials. Biocompatibility may, in principle, be achieved by adhesion of cells and extracellular material, e.g., matrix material (fouling), frequently followed by encapsulation; or biocompatibility may be achieved by prevention of adhesion of cells and extracellular material. As will be understood from the description elsewhere herein, the dialysis tube of the device for implantation may in particular be biocompatible by non-adherence of biomolecules on order to maintain optimal diffusion over the dialysis membrane. Suitable materials for the dialysis tube are specified herein below.

In an embodiment, the device further comprises a port as specified herein below providing access to the dialysis tubing from the bodily surface of the mammalian host and/or from a subcutaneous access element; thus, in an embodiment, the device is adapted for in situ replacement of the dialysis tube. In a further embodiment, the device further comprises a port as specified herein below providing a conduct from the lumen of the dialysis tubing to the bodily surface of the mammalian host and/or to a subcutaneous access element; thus, in an embodiment, the device is adapted for in situ replacement of the content of the dialysis tube. In a further embodiment, the device is adapted for in situ replacement of the dialysis tube. In an embodiment, the device further comprises at least one sensor element as specified herein below accessible via the port and extending at least partially into the lumen of the dialysis tubing.

In an embodiment, the device further comprises an implantable first guiding system at least partially guiding the dialysis tube in a pre-specified direction, e.g., through a tissue, as specified herein below. In an embodiment, the first guiding system guides, optionally in conjunction with the port, the dialysis tube through all layers of tissue into a body cavity.

In particular in cases where a considerable volume of the lumen and/or length of dialysis tube is desirable, it may be envisaged that the device comprises more than one dialysis tube as specified and/or more than one first guiding system, e.g., two, five, ten, 25, or even 50 dialysis tubes and/or first guiding systems. Thus, in an embodiment, the device for implantation comprises of from 2 to 50, in a further embodiment of from 3 to 25, in a further embodiment, of from 5 to 15 dialysis tubes and/or first guiding systems. In such case, the dialysis tubes and/or first guiding systems may be spatially oriented in any order deemed appropriate by the skilled person for the intended use; e.g., dialysis tubes and/or first guiding systems may be oriented in essentially the same direction, e.g., in a parallel orientation, or may be arranged in a radiant order, which may be three-dimensionally radiant or 2-dimensionally radiant. In an embodiment, each first guiding system guides up to five, in an embodiment up to four, in a further embodiment up to three, in a further embodiment up to two, in a further embodiment guides one dialysis tube. In another embodiment, the device may comprise one dialysis tube, which may be spatially oriented to fold back to the port of the device at least once, thus inducing a folded structure; or the first guiding system(s) may enforce a spiral or meandering arrangement. In an embodiment, the dialysis tubes and/or first guiding systems are arranged, in an embodiment fixed, in an arrangement ensuring that the dialysis tubes are contacted by bodily fluid in the body cavity over at least 50%, in an embodiment at least 75%, in a further embodiment at least 85%, in a further embodiment at least 90% of their surface. Thus, in an embodiment, at least the dialysis tube of the device is at least partially implanted intraperitoneally, subcutaneously, intraarterially, or intravenously, in an embodiment intraperitoneally, in a further embodiment intraabdominal.

In an embodiment, the device for implantation is a transcutaneous implantable device, i.e., at least part of the structure of the device extends through the skin of the mammalian host. In an embodiment, the part of the structure extending through the skin is a transcutaneous port as specified herein below.

In an embodiment, the device for implantation is implanted as a complete device, i.e., comprising all features and elements as specified. In a further embodiment, the port of the device including at least part of the first guiding system(s), is implanted first and the dialysis tube(s) are introduced at a later point in time, e.g., after healing in. It is, however, also envisaged that the port is implanted first and that the first guiding system(s), optionally including the dialysis tube(s), are mounted later, e.g., after healing in; said proceeding may, e.g., be particularly useful where a radial arrangement of the first guiding systems and/or dialysis tubes is desired.

The term “mammalian host,” as used herein, relates to an mammalian animal, in an embodiment a livestock, such as a cattle, a pig, a horse, a sheep, or a goat; a pet, such as a rabbit, a guinea pig, or a hare; a companion animal, such as a cat or dog; or a laboratory animal, such as a mouse or a rat. In a further embodiment, the mammalian host is a human.

The term “dialysis tube” is in principle known to the skilled person as relating to a device comprising a dialysis membrane enclosing a lumen. As referred to herein, the dialysis tube has an outer diameter of from 20 μm to 600 μm. In an embodiment, the dialysis tube has an outer diameter of from 100 μm to 550 μm, in an embodiment of from 200 μm to 500 μm, in a further embodiment of from 300 μm to 500 μm, in a further embodiment of from 400 μm to 500 μm. In an embodiment, the dialysis tube has an outer diameter of 500 μm±10%. As is understood by the skilled person, the length of the dialysis tube will depend on the application envisaged, in particular e.g., on the number of cells deemed desirable for the application, on the cell density in the dialysis tube, and on the diameter of the dialysis tube. Typically, the dialysis tube may have a length of from 10 cm to 2000 cm, in an embodiment of from 20 cm to 1000 cm, in a further embodiment of from 50 cm to 800 cm, in particular in case the dialysis tube has an outer diameter of about 500 μm. As is understood by the skilled person, the dialysis membrane of the dialysis tube is closed at the circumference of the dialysis tube, in an embodiment is continuous, and the dialysis tube in an embodiment has openings only at the ends end, of which one may be sealed before, and the other may be sealed after filling.

As detailed elsewhere herein, it may be envisaged to provide a multitude of dialysis tubes and/or specific spatial arrangements of the dialysis tube(s) to reduce overall size of the device. Thus, the device for implantation may comprise a multitude of dialysis tubes; in such case, the dialysis tubes may be spatially oriented in any order deemed appropriate by the skilled person for the intended use; e.g., the dialysis tubes may be oriented in essentially the same direction, e.g., in a parallel orientation, or may be arranged in a radiant order, which may be three-dimensionally radiant or 2-dimensionally radiant. In a further embodiment, the dialysis tube may be spatially oriented to fold back to the port of the device at least once, and thus may have a folded structure. It may also be envisaged that the dialysis tube has a spiral or meandering orientation. The dialysis tube may, however, also assume a random spatial arrangement, e.g., in in cases where there is only partial or no guidance by a first guiding system, or in cases where first guiding system and/or a port is/are absent. In an embodiment, the dialysis tube is covered at least partially with a biocompatible outer layer, in an embodiment with a hydrogel. Biocompatible layers such as hydrogels are known in the art. As is understood by the skilled person, biocompatible layers preventing deposition of biomaterial are particularly envisaged.

The term “lumen” of a dialysis tube is understood by the skilled person to relate to the volume of the dialysis tube essentially enclosed by the dialysis membrane; in an embodiment, other structural elements may contribute to delimitation of the lumen, such as fastening and/or sealing elements, e.g., sealing the dialysis tube at one or both ends. In an embodiment, the lumen of the dialysis tube has a volume of at least 1 mm³, in an embodiment at least 10 mm³, in a further embodiment at least 100 mm³, in a further embodiment at least 1 cm³, in a further embodiment at least 2.5 cm³, in a further embodiment at least 5 cm³. In a further embodiment, the lumen of the dialysis tube has a volume of from 1 mm 3 to 5 cm³, in an embodiment of from 10 mm 3 to 2.5 cm³, in a further embodiment of from 100 mm 3 to 1.5 cm³. In an embodiment, the lumen of the dialysis tube is accessible from a bodily surface of a mammalian host via the port.

The term “dialysis membrane,” as used herein, relates to a semipermeable membrane having a molecular weight cutoff of from 25 kDa to 150 kDa and comprising at least one material selected from the group consisting of polyethersulfone, polyarylethersulfone, polyethylene, polytetrafluoroethylene, polyvinylidene fluoride and polypropylene. As used herein, the term “molecular weight cutoff” relates to the lowest molecular weight of a solute which is retained by at least 90% by a dialysis membrane; i.e., solutes having at least the molecular weight of the cutoff value are retained by 90% or more by the dialysis membrane, whereas solutes with a molecular weight with lower than the cutoff value are retained by less than 90%. The skilled person is aware that not all molecules of a given molecular weight have the same form, size, and diffusion properties and that, therefore, the molecular weight cutoff value is an average value. In accordance, there is only a rough correlation between the molecular weight cutoff of a dialysis membrane and the diameter of its pores; e.g., for a molecular weight cutoff of 100 kDa, pore size in an embodiment is in the range of 9 to 10 nm. Methods for determining the molecular weight cutoff of a dialysis membrane are known in the art. Typically, the value of the molecular weight cutoff of a dialysis membrane is the value provided by the manufacturer. In an embodiment, the molecular weight cutoff of the dialysis membrane is adjusted by the skilled person in dependence on the therapeutic compound produced in the dialysis tube. In an aspect, it was found that it may be envisaged to select the molecular weight cutoff of the dialysis membrane to allow diffusion of nutrients and peptidic growth factors, as well as of the therapeutic compound over the dialysis membrane, while in an embodiment excluding components of the immune system, in particular immunoglobulins, from the lumen of the dialysis tube may be desirable. Thus, the dialysis membrane in an embodiment has a molecular weight cutoff of at least 25 kDa, in a further embodiment at least 50 kDa, in a further embodiment at least 80 kDa; and/or the dialysis membrane in an embodiment has a molecular weight cutoff of at most 125 kDa, in a further embodiment at most 110 kDa. Thus, in an embodiment, the dialysis membrane has a molecular weight cutoff of from 25 kDa to 125 kDa, in an embodiment of from 40 kDa to 110 kDa, in a further embodiment of from 50 kDa to 100 kDa, in a further embodiment of from 80 kDa to 100 kDa, in a further embodiment of 100 kDa±10%, in a further embodiment of 100 kDa.

The dialysis membrane comprises at least one material selected from the group consisting of polyethersulfone, polyarylethersulfone, polyethylene, polytetrafluoroethylene, polyvinylidene fluoride and polypropylene; the polyarylethersulfone, in an embodiment, is polyethersulfone, polyphenylenesulfone (PPSU), or polysulfone (PSU). Thus, the dialysis membrane may comprise one or more of the aforesaid materials, or a mixture thereof. In an embodiment, the dialysis membrane consists of at least one material selected from the group consisting of polyethersulfone, polyarylethersulfone, polyethylene, polytetrafluoroethylene, polyvinylidene fluoride and polypropylene, or of a mixture thereof. In a further embodiment, the dialysis membrane consists of polyethersulfone, polyarylethersulfone, polyethylene, polytetrafluoroethylene, polyvinylidene fluoride or polypropylene. Polyethersulfone (Poly(oxy-1,4-phenylenesulfonyl-1,4-phenylene)), CAS No. 25667-42-9), polyphenylenesulfone ((1,1′-Biphenyl)-4,4′-diol, polymer with 1,1′-sulfonylbis(4-chlorobenzene)) CAS No. 25608-64-4; polysulfone (Poly(oxy-1,4-phenylenesulfonyl-1,4-phenyleneoxy-1,4-phenylene(1-methylethylidene)-1,4-phenylene), CAS No. 25135-51-7; polyethylene (polyethene, CAS No. 9002-88-4), polytetrafluoroethylene (Poly(1,1,2,2-tetrafluoroethylene), CAS No. 9002-84-0), polyvinylidene fluoride (Poly(1,1-difluoroethylene), CAS No. 24937-79-9) and polypropylene (Poly(l-methylethylene), CAS No. 9003-07-0) are known in the art, as are methods for producing dialysis membranes from these materials. In an embodiment, the material of the dialysis membrane is selected to be biocompatible as well as compatible with the therapeutic compound, i.e., in an embodiment, does not cause denaturation and/or aggregation of the therapeutic compound; the aforesaid property can be tested, e.g., as shown herein in Example 2.

The term “therapeutic composition” as used herein, relates to any and all compositions comprising (i) a plurality of cells secreting a therapeutic compound and (ii) a matrix material, both as specified herein below. In an embodiment, the therapeutic composition is a liquid at the time of filling the dialysis tube and/or is solid or semisolid, e.g., a gel, at the time of implantation or at latest 1 day after implantation. The therapeutic composition may comprise further compounds and components in addition to those specified above. Typical compounds optionally additionally present may be salts, e.g., salts aiding in gelling of the matrix material, buffers, antibiotics, inflammation inhibitors, water, organic solvents, and the like, which are in an embodiment selected so as not to affect the biological activity of the cells secreting a therapeutic compound, in particular secretion of the therapeutic compound, and/or cell viability. In an embodiment, the therapeutic composition comprises at least one further cell type, in an embodiment feeder cells and/or modulator cells, e.g., cells activating secretion of the therapeutic compound. In an embodiment, the therapeutic composition comprises at least insulin secreting cells, in a further embodiment comprises insulin secreting cells and glucagon secreting cells.

In an embodiment, the therapeutic composition comprises at least 10³, in an embodiment at least 10⁵, in a further embodiment at least 10⁶, in a further embodiment at least 10⁷, in a further embodiment at least 10⁸, in a further embodiment at least 10⁹, cells secreting a therapeutic compound. As referred to herein, the indicated number of cells comprised in the therapeutic composition, as well as values for the volume of the lumen of the dialysis tube(s), refer to the total volume of therapeutic composition and/or lumen available per device; i.e., in case the device for implantation comprises more than one dialysis tube, the number of cells comprised in the therapeutic composition is the number of cells comprised in all dialysis tubes added up. In an embodiment, the density of cells secreting a therapeutic compound in the therapeutic composition is of from 10⁴/ml to 10⁹/ml, in an embodiment of from 10⁵/ml to 10⁸/ml. In an embodiment, at least 50% of cells secreting a therapeutic compound remain viable for a period of at least 1 week, in an embodiment at least one month, in a further embodiment at least three months, in a further embodiment at least six months, in a further embodiment at least one year, in the therapeutic composition in the implanted device for implantation. As the skilled person understands, the composition of the therapeutic composition may change over time after implantation, e.g., nutrients and growth factors may diffuse into the therapeutic composition, and/or therapeutic compounds may diffuse out of the therapeutic composition. Thus, in case of doubt, the composition of the therapeutic composition as specified is the composition at the time before or at implantation.

The term “plurality,” as used herein, relates to a number of more than one, i.e., at least two, in an embodiment at least three, in a further embodiment at least five, in a further embodiment at least ten. In particular in the context of a plurality of cells, the term plurality in an embodiment relates to a number of at least 10³, in an embodiment at least 10⁵, in a further embodiment at least 10⁶, in a further embodiment at least 10⁷, in a further embodiment at least 10⁸, cells.

The term “therapeutic compound,” as used herein, includes any and all compounds produced and secreted by a cell secreting a therapeutic compound as specified herein below. In an embodiment, the therapeutic composition is a compound having a molecular weight of less than 150 kDa, in an embodiment less than 150 kDa, in a further embodiment less than 100 kDa. As used herein, the term “molecular weight” of a therapeutic compound relates to the molecular weight of the compound as it is secreted by the cells secreting a therapeutic compound; thus, the molecular weight is the molecular weight of, e.g., the tetrameric compound in case the therapeutic compound is secreted as a tetramer. In an embodiment, the therapeutic compound is a compound for which a basal level is required to be produced in a mammalian host. In an embodiment, the therapeutic compound is a biological macromolecule, such as a polypeptide, a polynucleotide, a polysaccharide, and the like, in an embodiment is a polypeptide. In an embodiment, the therapeutic compound is selected from the group consisting of insulin, glucagon, Factor IX, human growth hormone (huGH), Calcitonin, Glucagon-like peptide (GLP-1) and agonists, Amylin, levodopa, somatostatin, alpha1-antitrypsin (AAT), interleukins, and interferons. In a further embodiment, the therapeutic compound is insulin or an insulin analog, in an embodiment is human insulin or an analog of human insulin or a human or humanized insulin analog, in a further embodiment is human insulin.

The term “cells secreting a therapeutic compound” includes each and every eukaryotic cell secreting a therapeutic compound as specified herein above. In an embodiment, said cells are mammalian cells, in a further embodiment human cells. In an embodiment, the cells are from the same species as the mammalian host. Said cells may be, but do not have to be, autologous cells. In an embodiment, the cells secreting a therapeutic compound are pancreatic beta-cells, in an embodiment differentiated from stem cells, in an embodiment from a stem cell line, in a further embodiment from a pluripotent stem cell line. Preferably, said stem cells were not generated by destruction of an embryo, in particular a human embryo. In an embodiment, the cells secreting a therapeutic compound are comprised in pancreatic islets or pancreatic islet-like clusters. In an embodiment, the cells secreting a therapeutic compound are comprised in the therapeutic composition as aggregates comprising at least 10, in an embodiment at least 100, in a further embodiment at least 1000 cells. In a further embodiment, the cells secreting a therapeutic compound are comprised in the therapeutic composition as aggregates having an average diameter of from 100 μm to 400 μm, in an embodiment of from 200 μm to 300 μm.

The term “matrix material” includes each and every material deemed suitable by the skilled person for embedding and maintaining viability of cells secreting a therapeutic compound. Thus, the matrix material, in an embodiment, is adapted to permit diffusion of nutrients, including oxygen, and growth factors to said cells, and diffusion of the therapeutic compound from the cells. In accordance, matrix materials forming a semisolid matrix, e.g., a gel, may be used. Also, the matrix material is selected to not be deleterious to the cells secreting a therapeutic compound and/or the mammalian host. Appropriate matrix materials are known in the art. In an embodiment, the matrix material is selected from alginate, collagen, fibrin, extracellular matrix material, synthetic hydrogel, and acrylate, in an embodiment the matrix material is alginate. Extracellular matrix materials are known in the art; e.g., an extracellular matrix material secreted by Engelbreth-Holm-Swarm (EHS) mouse sarcoma cells is marketed under the trade name Matrigel®.

The term “port,” as used herein, relates to any appliance installable at least partially under the skin of the mammalian host and providing access to the dialysis tube(s) of the device for implantation. Thus, the port comprises a first connecting element connecting the dialysis tube(s) to a fixing element of the port, wherein said fixing element fixes the port within or under the skin of the mammalian host. The first connecting element may provide fluid access to the lumen of the dialysis tube(s) and/or may hold the dialysis tube(s) in place after implantation. As is understood from the description herein above, the first connecting element may provide for a releasable fastening engagement between the dialysis tube(s) and the fixing element of the port. Thus, the port may be a subcutaneous port providing fluid connection to the lumen of the dialysis tube. In a further embodiment, the port is a transcutaneous port, i.e., a transcutaneously implantable port; in such case, the port may further comprise a second connecting element connecting the fixing element of the port to an extracorporeal connecting head. Said extracorporeal connecting head may be a sealing element and/or may comprise one or more connectors connecting the optional sensors extending into the lumen of the dialysis tube to a signal transducer unit. In an embodiment, the port is a port a described in EP 1 420 836 B1, which is herewith incorporated by reference, with the catheter adapted to function as the first guiding system as specified herein, and optionally additional elements adapted to perform functions a s specified herein.

The term “first guiding system,” as used herein, relates to a rigid or semi-rigid element of the port extending, after implantation, at least partially through a tissue of the mammalian host. In an embodiment, the first guiding system has a U-profile or is tube-shaped. Thus, in an embodiment, the first guiding system at least partially covers the dialysis tube(s), wherein in an embodiment the first guiding system comprises a multitude of holes, which may have any shape deemed appropriate by the skilled person, e.g., round, elliptic, slot-shaped, or irregularly shaped. In an embodiment, said holes have an equivalent diameter of at least 0.05 μm, in an embodiment at least 0.5 μm, in a further embodiment at least 5 μm, in a further embodiment at least 50 μm, in a further embodiment at least 150 μm. In an embodiment, the first guiding system comprises, in an embodiment consists of, a material as specified herein above for the dialysis membrane, in a further embodiment comprises, in a further embodiment consists of, polytetrafluoroethylene (PTFE). It is, however, also envisaged that the first guiding system comprises, in an embodiment consists of, polyurethane (PUR).

The term “sensor element,” as used herein includes any and all sensing elements deemed appropriate by the skilled person to provide information on the state of the cells secreting a therapeutic compound comprised in the lumen of the dialysis tube(s). Thus, in an embodiment, the sensor element comprises at least one of an oxygen sensor, a glucose sensor, a pH-sensor, a lactate sensor, an insulin sensor, a C-peptide sensor, an inflammatory biomarker sensor, and/or a sensor for a marker of cell viability. Appropriate sensors are known in the art; in an embodiment, the sensor element is a microsensor, in a further embodiment an enzymatic microsensor.

Advantageously, it was found in the work underlying this disclosure that the dialysis membrane materials, molecular weight cutoffs, and outer diameter of a dialysis tube as specified after step b), alone or in combination, is or are particularly useful for maintenance of cells secreting a therapeutic compound as implants.

The definitions made above apply mutatis mutandis to the following. Additional definitions and explanations made further below also apply for all embodiments described in this specification mutatis mutandis.

This disclosure further relates to a method of producing a device for implantation according to this disclosure comprising:
a) filling the dialysis tube with cells secreting a therapeutic compound and a matrix material;
b) at least temporarily sealing at least one end of the dialysis tube; and
c) thereby, producing a device for implantation.

The method of the present in this disclosure, preferably, is an in vitro method. Moreover, it may comprise steps in addition to those explicitly mentioned above. Moreover, one or more of said steps may be assisted or performed by automated equipment. Exemplary additional steps may relate, e.g., to providing cells secreting a therapeutic compound for step a), sealing the second end of the dialysis tube in step b), and/or mounting the dialysis tube onto a port as specified herein above after step b). In accordance with the above, the method may further comprise admixing feeder and/or modulator cells or other compounds to the cells secreting a therapeutic compound before filling the dialysis tube.

The term “filling” is understood by the skilled person. In an embodiment, the dialysis tube is completely filled with cells secreting a therapeutic compound and a matrix material or its precursor(s), i.e., with a therapeutic composition as specified herein above. Thus, in an embodiment, there is no void volume or gas trapped in the dialysis tube. Filling may also comprise a step of polymerizing the matrix material; thus, the therapeutic composition may be filled into the dialysis tube as a liquid, i.e., as a suspension of cells in the matrix material or its precursor(s), and the matrix material or its precursor(s) may be induced or left to polymerize in the dialysis tube. In the context of filling the dialysis tube as specified herein, the term “polymerization” is to be understood in a broad sense relating to any chemical or physical process causing at least partial solidification of the matrix material; thus, polymerization may involve formation of covalent bonds, ionic bonds, and/or van der Waals interactions between molecules of the matrix material or its precursor(s). E.g., in case the matrix material is alginate, polymerization may be induced by contacting the filled dialysis tube with a solution comprising divalent cations, in an embodiment alkaline earth metal cations, in particular calcium ions and/or barium ions, which diffuse into the matrix material and cause gelling or solidification. In accordance, the dialysis tube(s) may be washed, in an embodiment for extended periods of time such as 15 min, 30 min, or 1 hour, to remove polymerization reagents and/or non-polymerized precursors of the matrix material.

This disclosure also relates to a port system, comprising:
I) a device for implantation comprising a port according to this disclosure and/or a device for implantation comprising a port obtained or obtainable according the method of producing a device for implantation; and
II) an extracorporeal connecting head.

The port system comprises the components specified, which are described herein in the context of the device for implantation; the port system may, however, comprise further optional components, e.g., a signal transducer unit as specified herein below or accessory components, e.g., enabling access to the dialysis tube(s) and/or their lumen(s), e.g., a membrane, a removable further fixing element, and the like. In an embodiment, the port is a transcutaneous implantable port and comprises a second connecting element connecting the port unit to the extracorporeal connecting head. In a further embodiment, the extracorporeal connecting head can be fastened to the device for implantation by a releasable fastening engagement. The port may, however, also be a subcutaneous port and the extracorporeal connecting head may be fixed to the port by indirect means, e.g., magnetic force. In an embodiment, the extracorporeal connecting head at one end is adapted to be positioned on the skin of the mammalian host.

The term “signal transducer unit” is understood by the skilled person to relate to provide means for transduction of a signal of a sensor element to an appropriate evaluation device, e.g., a measuring, monitoring, and/or display device. In an embodiment, the signal transducer unit is adapted to output at least one signal obtained by a sensor element. Suitable signal transducer units are known to the skilled person and include simple output units such as a connector cable or connector plug. A signal transducer unit may, however, also be an interface to an evaluation device, wherein said interface may be any kind of means of transferring data, including, e.g., cable connections like USB, wireless connections like wireless LAN, Bluetooth, and the like, or indirect connections such as data transfer by instant messaging, email, or the like.

In an embodiment, the port system of this disclosure is part of a monitoring system comprising a port system as specified herein above and an evaluation device. As will be understood by the skilled person, the evaluation device may be comprised in the same housing as the port system, e.g., as an evaluation unit comprised in the extracorporeal connecting head, or may be a separate device, e.g., a handheld device. In an embodiment, the evaluation device comprises a microprocessor programmed to receive a signal, e.g., output data, from a signal transducer unit and to perform logical operations providing an evaluation of said output data. Evaluation of output data may comprise, e.g., correcting data for values measured in one or more control sensors, statistical calculations, e.g., calculating means of two or more parallel detection reactions, correcting data measurement errors, comparing output data to reference values, compiling data in a list, and the like. In an embodiment, the evaluation device further comprises a data storage unit. In a further embodiment, said data storage unit comprises reference values, e.g., in a reference value data base. Moreover, in an embodiment, the data storage unit is adapted to store output data received from a sensor element of this disclosure, as specified above.

This disclosure also relates to a cell secreting a therapeutic compound for use in treating disease by implantation of said cell in a device for implantation according to this disclosure and/or a device obtained or obtainable according to the method of producing a device for implantation according to this disclosure.

This disclosure also relates to a cell secreting a therapeutic compound for use in treating diabetes, Hemophilia B, huGH deficiency, osteoporosis, Parkinson's disease, acromegaly, an endocrine tumor, alpha1-antitrypsin-deficiency, an infectious disease, an autoimmune disease, cancer, or chronic virus infection by implantation of said cell in a device for implantation according to this disclosure and/or a device obtained or obtainable according to the method of producing a device for implantation according to this disclosure.

This disclosure also relates to a method of treating and/or preventing disease comprising implanting a device for implantation of this disclosure and/or a device obtained or obtainable according to the method of producing a device for implantation according to this disclosure.

The diseases and disorders referred to herein are well-known in the art. As is understood by the skilled person, the underlying cause of the disease in an embodiment is a deficit or the absence of a compound, in particular a polypeptide, from the body of the affected mammalian host; e.g., in hemophilia, one or more coagulation factors may be produced at an insufficient amount by the mammalian host; in diabetes, the amount of insulin may be insufficient, and the like. Diabetes, in an embodiment, is diabetes type I.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50%, at least 60%, at least 70%, at least 80%, or at least 90% of the subjects of a given cohort or population.

The term “preventing” refers to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to this disclosure. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e., without preventive measures according to this disclosure, would develop a disease or disorder as referred to herein. Whether a portion is statistically significant can be determined without further ado by the person skilled in the art using various well known statistic evaluation tools discussed elsewhere in this specification.

In view of the above, the following embodiments are particularly envisaged:

Embodiment 1: A device for implantation into a mammalian host, said device comprising:
a) a dialysis tube with a lumen and a dialysis membrane enclosing said lumen, wherein
   i) the dialysis tube has an outer diameter of from 20 μm to 600 μm; and
   ii) the dialysis membrane has a molecular weight cutoff of from 25 kDa to 150 kDa and comprises at least one material selected from the group consisting of polyethersulfone, polyarylethersulfone, polyethylene, polytetrafluoroethylene, polyvinylidene fluoride, and polypropylene; and
b) a therapeutic composition disposed within the lumen of the dialysis tube, wherein said therapeutic composition comprises a plurality of cells secreting a therapeutic compound and a matrix material embedding said cells secreting a therapeutic compound, wherein the matrix material is selected from alginate, collagen, fibrin, extracellular matrix material, synthetic hydrogel, and acrylate, in an embodiment the matrix material is alginate.

Embodiment 2: The device of embodiment 1, wherein the dialysis tube has an outer diameter of from 100 μm to 500 μm, in an embodiment of from 200 μm to 550 μm, in a further embodiment of from 300 μm to 500 μm, in a further embodiment of from 400 μm to 500 μm.

Embodiment 3: The device of embodiment 1 or 2, wherein the dialysis tube has a length of from 10 cm to 2000 cm, in an embodiment of from 20 cm to 1000 cm, in a further embodiment of from 50 cm to 800 cm.

Embodiment 4: The device of any one of embodiments 1 to 3, wherein the dialysis tube assumes a meandering, spiral, or radiant form in the body of the mammalian host.

Embodiment 5: The device of any one of embodiments 1 to 4, wherein the dialysis tube is covered at least partially with a biocompatible outer layer, in an embodiment with a hydrogel.

Embodiment 6: The device of any one of embodiments 1 to 5, wherein the lumen of the dialysis tube has a volume of at least 1 mm³, in an embodiment at least 10 mm³, in a further embodiment at least 100 mm³, in a further embodiment at least 1 cm³, in a further embodiment at least 2.5 cm³, in a further embodiment at least 5 cm³.

Embodiment 7: The device of any one of embodiments 1 to 6, wherein the lumen of the dialysis tube has a volume of from 1 mm 3 to 5 cm³, in an embodiment of from 10 mm 3 to 2.5 cm³, in a further embodiment of from 100 mm 3 to 1.5 cm³.

Embodiment 8: The device of any one of embodiments 1 to 7, wherein said therapeutic composition comprises at least 10³, in an embodiment at least 10⁵, in a further embodiment at least 10⁶, in a further embodiment at least 10⁷, in a further embodiment at least 10⁸, in a further embodiment at least 10⁹, cells secreting a therapeutic compound.

Embodiment 9: The device of any one of embodiments 1 to 8, wherein said cells secreting a therapeutic compound are comprised in said therapeutic composition as aggregates comprising at least 10, in an embodiment at least 100, in a further embodiment at least 1000, cells.

Embodiment 10: The device of any one of embodiments 1 to 9, wherein said cells secreting a therapeutic compound are comprised in said therapeutic composition as aggregates having an average diameter of from 100 μm to 400 μm, in an embodiment of from 200 μm to 300 μm.

Embodiment 11: The device of any one of embodiments 1 to 10, wherein said therapeutic composition comprises at least one further cell type, in an embodiment feeder cells and/or modulator cells.

Embodiment 12: The device of any one of embodiments 1 to 11, wherein the dialysis membrane has a molecular weight cutoff of from 25 kDa to 125 kDa, in an embodiment of from 40 kDa to 110 kDa.

Embodiment 13: The device of any one of embodiments 1 to 12, wherein the dialysis membrane has a molecular weight cutoff of from 50 kDa to 100 kDa.

Embodiment 14: The device of any one of embodiments 1 to 13, wherein the dialysis membrane has a molecular weight cutoff of from 80 kDa to 100 kDa.

Embodiment 15: The device of any one of embodiments 1 to 14, wherein the dialysis membrane has a molecular weight cutoff of 100 kDa.

Embodiment 16: The device of any one of embodiments 1 to 16, wherein the dialysis membrane consists of a material selected from the group consisting of polyethersulfone, polyarylethersulfone, polyethylene, polytetrafluoroethylene, polyvinylidene fluoride, and polypropylene.

Embodiment 17: The device of any one of embodiments 1 to 16, wherein said device further comprises an implantable first guiding system at least partially guiding the dialysis tube through a subcutaneous tissue.

Embodiment 18: The device of embodiment 17, wherein the first guiding system at least partially covers the dialysis tube, wherein in an embodiment the first guiding system comprises a multitude of holes with an equivalent diameter of at least 0.05 μm, in an embodiment at least 0.5 μm, in a further embodiment at least 5 μm, in a further embodiment at least 50 μm, in a further embodiment at least 150 μm.

Embodiment 19: The device of any one of embodiments 1 to 18, wherein said device further comprises a port providing access to the dialysis tubing from the bodily surface of the mammalian host and/or from a subcutaneous access element.

Embodiment 20: The device of embodiment 19, wherein said port is a transcutaneously implantable port.

Embodiment 21: The device of embodiment 19 or 20, wherein the port provides a conduct from the lumen of the dialysis tubing to the bodily surface of the mammalian host.

Embodiment 22: The device of any one of embodiments 19 to 21, wherein said port comprises a fixing element fixing the port within or under the skin of the mammalian host.

Embodiment 23: The device of any one of embodiments 19 to 22, wherein the port comprises a first connecting element connecting the dialysis tubing to the port.

Embodiment 24: The device of any one of embodiments 19 to 23, wherein said port comprises a second connecting element connecting the fixing element of the port to an extracorporeal connecting head.

Embodiment 25: The device of any one of embodiments 19 to 24, herein the lumen is accessible from a bodily surface of a mammalian host via the port.

Embodiment 26: The device of any one of embodiments 1 to 25, wherein at least the dialysis tube of the device is at least partially implanted intraperitoneally, subcutaneously, intraarterially, or intravenously, in an embodiment intraperitoneally.

Embodiment 27: The device of any one of embodiments 1 to 26, wherein the device further comprises at least one sensor element accessible via the port and extending at least partially into the lumen of the dialysis tubing.

Embodiment 28: The device of any one of embodiments 1 to 27, wherein said sensor element comprises at least one of an oxygen sensor, a glucose sensor, a pH-sensor, a lactate sensor, an insulin sensor, a C-peptide sensor, an inflammatory biomarker sensor, and/or a sensor for a marker of cell viability.

Embodiment 29: The device of any one of embodiments 1 to 28, wherein said device is adapted for in situ replacement of said dialysis tube.

Embodiment 30: The device of any one of embodiments 1 to 29, wherein said device is adapted for in situ replacement of the content of the dialysis tube.

Embodiment 31: The device of any one of embodiments 1 to 31, wherein the mammalian host is a human, a laboratory animal, a livestock, a pet, or a companion animal, in an embodiment is a human.

Embodiment 32: The device of any one of embodiments 1 to 32, wherein the therapeutic compound is selected from the group consisting of insulin, glucagon, Factor IX, human growth hormone (huGH), Calcitonin, Glucagon-like peptide (GLP-1) and agonists, Amylin, levodopa, somatostatin, alpha1-antitrypsin (AAT), interleukins, and interferons.

Embodiment 33: The device of any one of embodiments 1 to 33, wherein the therapeutic compound is insulin or an insulin analog, in an embodiment is human insulin.

Embodiment 34: The device of any one of embodiments 1 to 34, wherein the cells secreting a therapeutic compound are mammalian cells, in an embodiment are human cells.

Embodiment 35: The device of any one of embodiments 1 to 34, wherein the cells secreting a therapeutic compound are pancreatic beta-cells, in an embodiment differentiated from stem cells, in an embodiment from a stem cell line, in a further embodiment from a pluripotent stem cell line.

Embodiment 36: The device of any one of embodiments 1 to 35, wherein the cells secreting a therapeutic compound are comprised in pancreatic islets or pancreatic islet-like clusters.

Embodiment 37: A method of producing a device for implantation according to any one of embodiments 1 to 36 comprising:
a) filling the dialysis tube with cells secreting a therapeutic compound and a matrix material;
b) at least temporarily sealing at least one end of the dialysis tube; and
c) thereby, producing a device for implantation.

Embodiment 38: The method of embodiment 37, wherein the method further comprises polymerizing the matrix material and/or at least one precursor of the matrix material in the dialysis tube.

Embodiment 39: The method of embodiment 37 or 38, wherein said method further comprises washing the dialysis tube.

Embodiment 40: The method of any one of embodiments 37 to 39, wherein said device for implantation further comprises a port, in an embodiment as specified in any one of embodiments 20 to 26, and wherein the method further comprises fixing the dialysis tube to the port.

Embodiment 41: The method of any one of embodiments 37 to 40, further comprising admixing feeder and/or modulator cells to the cells secreting a therapeutic compound before filling the dialysis tube.

Embodiment 42: A port system, comprising:
I) a device for implantation comprising a port according to any one of embodiments 20 to 26 and/or a device for implantation obtained or obtainable according to embodiment 40; and
II) an extracorporeal connecting head.

Embodiment 43: The port system of embodiment 42, wherein the port is a transcutaneous implantable port and comprises a second connecting element connecting the port unit to the extracorporeal connecting head.

Embodiment 44: The port system of embodiment 43, wherein the extracorporeal connecting head can be fastened to the device for implantation by a releasable fastening engagement.

Embodiment 45: The port system of any one of embodiments 42 to 44, wherein the extracorporeal connecting head at one end is adapted to be positioned on the skin.

Embodiment 46: A cell secreting a therapeutic compound for use in treating disease by implantation of said cell in a device for implantation according to any one of embodiments 1 to 36 and/or obtained or obtainable according to the method of any one of embodiments 37 to 41.

Embodiment 47: A cell secreting a therapeutic compound for use in treating diabetes, Hemophilia B, huGH deficiency, osteoporosis, Parkinson's disease, acromegaly, an endocrine tumor, alpha1-antitrypsin-deficiency, an infectious disease, an autoimmune disease, cancer, or chronic virus infection by implantation of said cell in a device for implantation according to any one of embodiments 1 to 36 and/or obtained or obtainable according to the method of any one of embodiments 37 to 41.

Embodiment 48: A method of treating and/or preventing disease comprising implanting a device for implantation according to any one of embodiments 1 to 36 and/or obtained or obtainable according to the method of any one of embodiments 37 to 41 into a mammalian subject in need thereof, thereby treating disease.

Embodiment 49: The method of treating and/or preventing disease of embodiment 48, wherein said disease is diabetes, Hemophilia B, huGH deficiency, osteoporosis, Parkinson's disease, acromegaly, an endocrine tumor, alpha1-antitrypsin-deficiency, an infectious disease, an autoimmune disease, cancer, or chronic virus infection.

All references cited in this specification are herewith incorporated by reference with respect to their entire disclosure content and the disclosure content specifically mentioned in this specification.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 is a schematic partial view of a device for implantation 10 comprising a dialysis tube 20 and a sealing means 45;

FIG. 2 is a schematic in situ view of a transcutaneous embodiment of the device for implantation 10;

FIG. 3 is a port system 200 with first guiding system 140;

FIG. 4 is a schematic view of a port system 200 comprising a first guiding system 140 with openings 170;

FIG. 5 is a schematic view of a port system 200 with sensor element 180;

FIG. 6 is a schematic in situ view of a transcutaneous embodiment of the device for implantation 10 containing a multitude of dialysis tubes 20;

FIG. 7A is an aggregation of insulin, measured as mean fluorescence intensity (MFI) in an Thioflavin T-assay, induced by membranes made of Polyvinylidene fluoride (PVDF), polyethersulfone (PES), and polyarylethersulfone (PAES);

FIG. 7B is an aggregation of insulin, measured as mean fluorescence intensity (MFI) in an Thioflavin T-assay, induced by membranes made of polytetrafluoroethylene (PTFE); and

FIG. 7C is an aggregation of insulin, measured as mean fluorescence intensity (MFI) in an Thioflavin T-assay, induced by membranes made of Polypropylene (PP), polyamide (PA), polyethylene (PE) and polyurethane (PU).

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

### Example 1: Device for Implantation with Optional Port

### 1.1 Device for Implantation

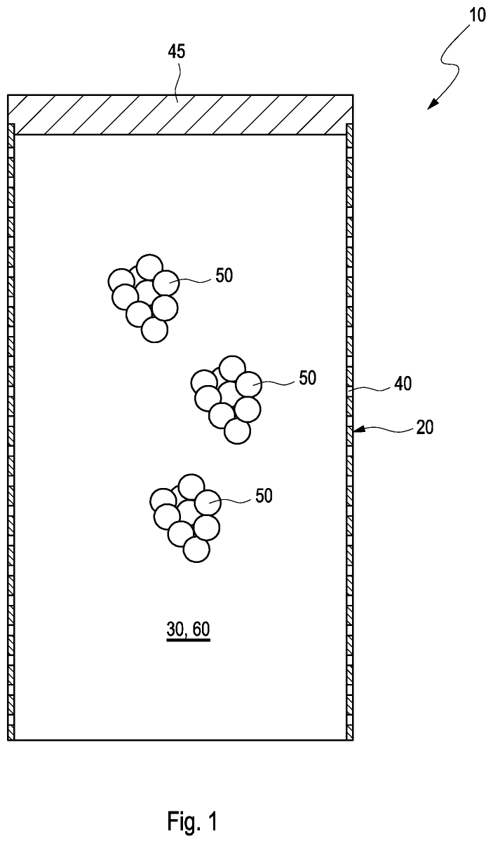

As shown in FIG. 1, the device for implantation 10 may comprise a dialysis tube 20 and a sealing means (also referred to as a “seal”) 45; as shown in FIG. 1, the device for implantation is open at the bottom part, e.g., in a state immediately filling and before sealing and/or integration into a port 70. The dialysis tube has a dialysis membrane 40 enclosing a lumen 30. Disposed within the lumen 30 are at least cells secreting a therapeutic compound 50 and a matrix material 60.

### 1.2 Transcutaneous Embodiment of the Device for Implantation

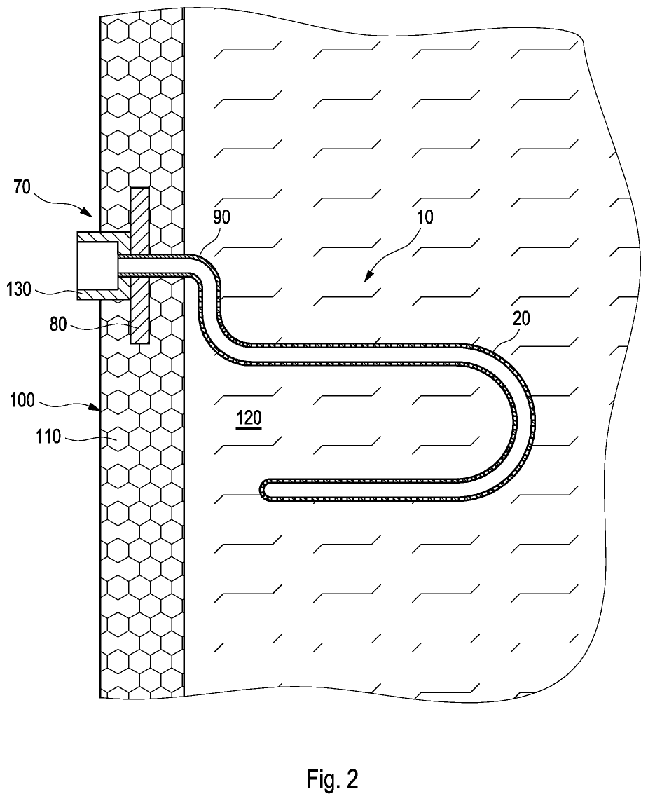

As shown in FIG. 2, the device for implantation 10 may be adapted for transcutaneous implantation. In accordance, the device for implantation 10 may comprise the dialysis tube 20 connected to a port 70 having a first connecting element 90, in turn connected to first fixing element 80, and transcutaneous portion 130. The transcutaneous portion 130 spans the skin 100 of the mammalian host. The first fixing element 80, located within the subcutaneous tissue, fixes the port 70 in the skin 100 of the mammalian host and, together with transcutaneous portion 130 provides access to the first connecting element and the dialysis tube 20 from the exterior of the body of the mammalian host. The port may provide fluid access to the lumen 30 of dialysis tube 20, as shown in FIG. 2; it is, however, also envisaged that dialysis tube 20 is sealed at both ends and that access via the port allows removal of the dialysis tube 20 and, in an embodiment, re-introduction of a fresh dialysis tube 20.

1.3 Port System with First Guiding System

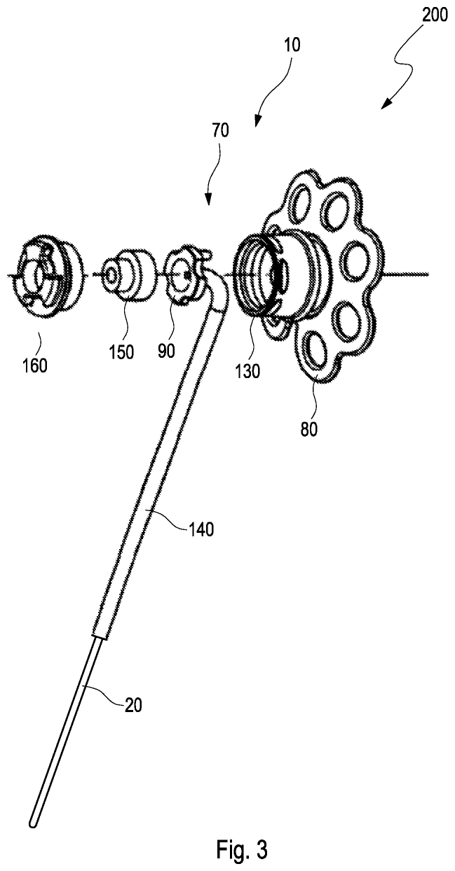

Shown in FIG. 3 is a port system 200 comprising a device for implantation 10 and an extracorporeal connecting head 160. The device for implantation comprises dialysis tube 20 and a port 70, wherein the port comprises a first fixing element 80, a transcutaneous portion 130, a first connecting element 90, and second connecting element 150. From first connecting element 90 protrudes first guiding system 140, which may guide dialysis tube, e.g., through subcutaneous tissue and/or may determine the spatial arrangement of dialysis tube 20. As shown in FIG. 3, first guiding system 140 only partially covers dialysis tube 20 over its longitudinal extension. It is, however, also envisaged that first guiding system 140 completely covers dialysis tube 20.

1.4 First Guiding System with Openings

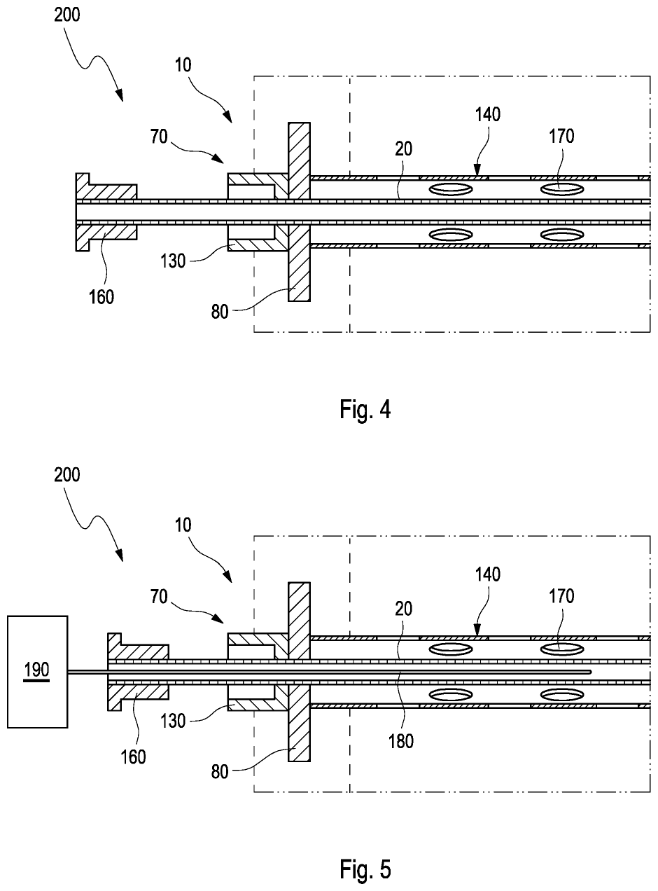

As described herein above and as shown in FIG. 4, first guiding system 140 may comprise openings 170, e.g., holes, which may, e.g., be introduced by laser ablation. Openings 170 in first guiding system 140 provide for free diffusion access to dialysis tube 20. The diameter of the first guiding system 140 may be substantially larger than the diameter of dialysis tube 20, as shown in FIG. 4; in accordance, first guiding system 140 may also accommodate a multitude of dialysis tubes 20 and/or of loops of a dialysis tube 20. The diameter of first guiding system 140 may, however, also be selected such that only one dialysis tube 20 can be accommodated within said first guiding system 140.

1.5 Port System with Sensor(s)

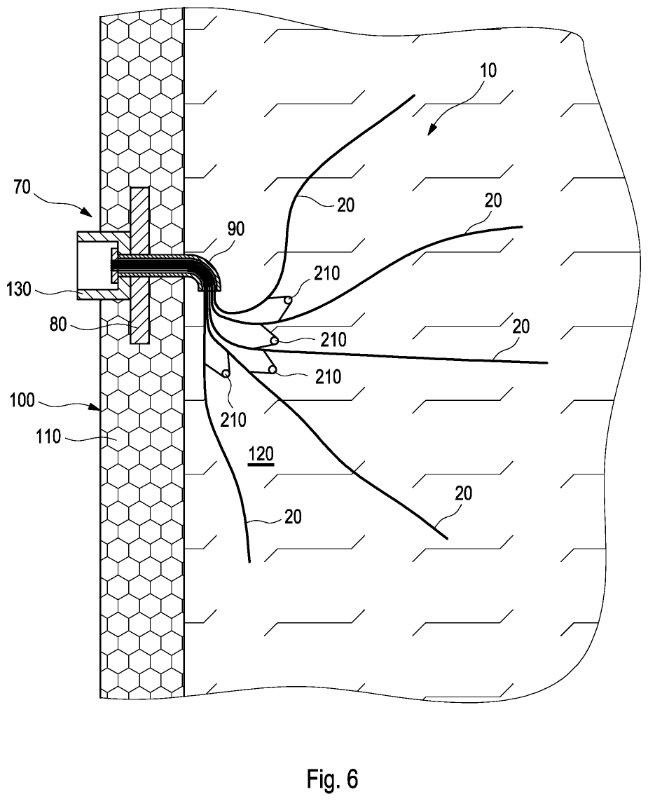

As shown in FIG. 5, port system 200, may comprise one or more sensors 180 extending into dialysis tube 20, permitting recording of information on the state of the cells secreting a therapeutic compound comprised therein. Sensor(s) 180 or their connectors may extend through port 70 and, optionally, through extracorporeal connecting head 160, and may be adapted for connection to a signal transducer unit 190.

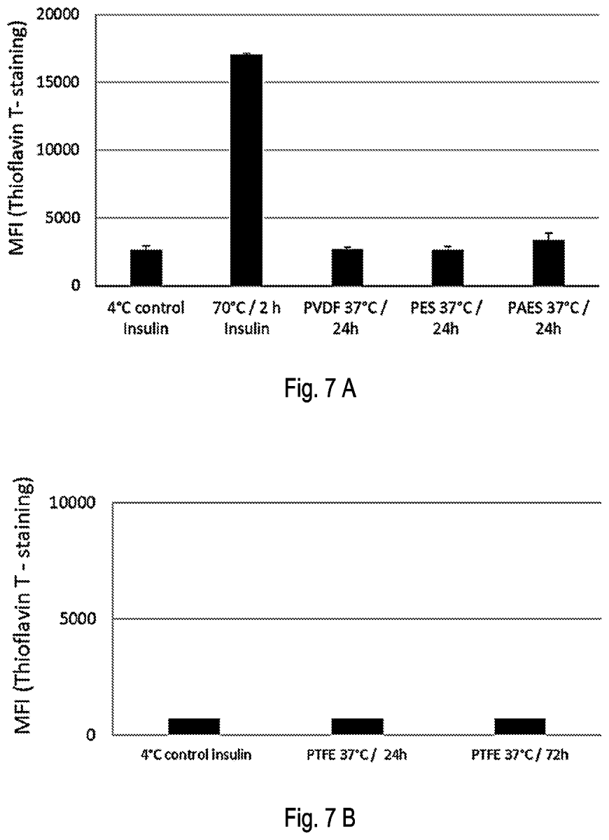

### 1.6 Shown Schematically in FIG. 6 is a Transcutaneous Embodiment of the Device for Implantation 10 Containing a Plurality of Dialysis Tubes 20.

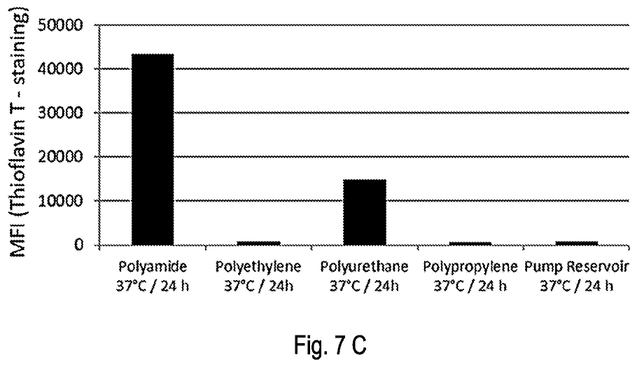

As shown in FIG. 6, the device for implantation 10 may be adapted for transcutaneous implantation. In accordance, the device for implantation 10 may comprise a plurality of dialysis tubes 20 connected to a port 70 having a first connecting element 90, in turn connected to first fixing element 80, and transcutaneous portion 130. The dialysis tubes 20 may be oriented in a radiant order and may be supported by one or more spreader elements 210, which may be configured to hold the dialysis tubes 20 in the desired orientation, e.g., by comprising a spring element. The transcutaneous portion 130 spans the skin 100 of the mammalian host. The first fixing element 80, located within the subcutaneous tissue, fixes the port 70 in the skin 100 of the mammalian host and, together with transcutaneous portion 130 provides access to the first connecting element and the dialysis tubes 20 from the exterior of the body of the mammalian host. The port may provide fluid access to the lumen 30 of dialysis tubes 20, as shown in FIG. 7; it is, however, also envisaged that dialysis tubes 20 are sealed at both ends and that access via the port allows removal of the dialysis tubes 20 and, in an embodiment, re-introduction of fresh dialysis tubes 20.

### Example 2: Insulin Compatibility of Different Membrane Materials

### 2.1 PVDF, PES and PAES

Sixteen PES (polyethersulfone) or PAES (polyarylethersulfone) membrane pieces (hollow fibers, CMA 65) were transferred to Eppendorf-tubes. Each piece was 3.0 cm in length and had a diameter of 0.5 mm (total surface area ca. 15 cm²). Polyvinylidene fluoride (PVDF) membrane strips were transferred to an Eppendorf-tube (15 cm² surface area). Each material was incubated with 1.5 ml insulin solution (1 mg/ml human insulin dissolved in phosphate buffered saline, PBS) at 37° C. with 300 rpm rotational agitation for 24 hours. As a positive control, insulin aggregation was induced in one sample by incubation at 70° C. for 2 h.

Thioflavin T (ThT) staining was performed to assess insulin aggregation/fibril formation. 50 μl of each sample was incubated with 150 μl PBS and 10 μl of ThT solution (450 μM) for 1 h in the dark. Mean fluorescence intensity (MFI) of the samples was analyzed using a Synergy4 plate reader (BioTek Instruments) at an excitation of λ=450 nm and an emission of λ=482 nm. Results are shown in FIG. 7A). A more than 2-fold increase of MFI relative to the non-aggregated insulin control (4° C.) indicates relevant aggregation which is undesirable. PVDF, PES and PAES did not induce insulin aggregation and were identified as insulin compatible materials.

### 2.2 PTFE

A 25 cm long polytetrafluoroethylene (PTFE) tubing (diameter: 0.4 mm) was connected to an Accu-Check Spirit Combo insulin pump. The pump reservoir was filled with human insulin solution (3 mg/ml, Insuman Infusat, Sanofi). Pump, connected tubing and an Eppendorf tube for sample collection were placed in an incubator at 37° C. Pump basal rate was set to 0.8 U/h (=8 μl/h). Additionally, 3 boli of 7 U (=70 μl) were delivered within 24 h. A total sample volume of ca. 400 μl was collected in an Eppendorf tube over 24 h.

The samples collected after 24 h and after 72 h were analyzed for insulin aggregation using ThT staining. 50 μl of each sample was incubated with 150 μl PBS and 10 μl of ThT solution (450 μM) for 1 h in the dark. Mean fluorescence intensity (MFI) of the samples was analyzed using a Synergy4 plate reader (BioTek Instruments) at an excitation of λ=450 nm and an emission of λ=482 nm. Results are shown in FIG. 7B). A more than 2-fold increase of MFI relative to the non-aggregated insulin control (4° C.) indicates relevant aggregation which is undesirable. PTFE did not induce insulin aggregation and was identified as an insulin compatible material.

### 2.3 PA, PE, PU, and PP

Tubing made either of polyamide (PA, ca. 30 cm²), polyethylene (PE, ca. 15 cm²), polyurethane (PU, ca. 30 cm²) or polypropylene (PP, ca. 30 cm²) were connected to Accu-Check Spirit Combo insulin pumps. The diameter of all tubing was 0.4 to 0.6 mm. The pump reservoirs were filled with human insulin solution (3 mg/ml, Humalog, Eli Lilly). Pumps, connected tubing and an Eppendorf tubes for sample collection were placed in an incubator at 37° C. Pump basal rate was set to 0.5 U/h (=5 μl/h). A total sample volume of ca. 120 μl was collected in each Eppendorf tube over 24 h. Insulin remaining in the insulin-compatible pump reservoir at the end of the experiment served as control.

The samples collected after 24 h were analyzed for insulin aggregation using ThT staining. 50 μl of each sample was incubated with 150 μl PBS and 10 μl of ThT solution (450 μM) for 1 h in the dark. Mean fluorescence intensity (MFI) of the samples was analyzed using a Synergy4 plate reader (BioTek Instruments) at an excitation of λ=450 nm and an emission of λ=482 nm. Results are shown in FIG. 7C). A more than 2-fold increase of MFI relative to the non-aggregated insulin control (pump reservoir) indicates relevant aggregation which is undesirable. PP and PE did not induce insulin aggregation and were identified as insulin compatible materials. PA and PU showed high MFI values indicating formation of insulin aggregates. PA and PU were identified as non-compatible materials.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

10 device for implantation
20 dialysis tube
30 lumen of dialysis tube
40 dialysis membrane
45 sealing means
50 cells secreting a therapeutic compound
60 matrix material
70 port
80 fixing element
90 first connecting element
100 skin of the mammalian host
110 subcutaneous tissue
120 body cavity, e.g., abdomen
130 transcutaneous portion
140 first guiding system
150 second connecting element
160 extracorporeal connecting head
170 opening (hole) in first guiding system
180 sensor element
190 signal transducer unit
200 port system
210 spreader element

What is claimed is:

1. A device for implantation into a mammalian host, comprising:
a) a dialysis tube with a lumen and a dialysis membrane enclosing the lumen, wherein:
i) the dialysis tube has an outer diameter of from 20 μm to 600 μm, and
ii) the dialysis membrane has a molecular weight cutoff of from 25 kDa to 150 kDa and comprises at least one material selected from the group consisting of polyethersulfone, polyarylethersulfone, polyethylene, polytetrafluoroethylene, polyvinylidene fluoride, and polypropylene; and
b) a therapeutic composition disposed within the lumen and comprising a plurality of cells configured for secreting a therapeutic compound, and a matrix material embedding said cells, wherein the matrix material is selected from the group consisting of alginate, collagen, fibrin, extracellular matrix material, synthetic hydrogel, and acrylate.

2. The device of claim 1, wherein the dialysis tube has an outer diameter of from 400 μm to 500 μm.

3. The device of claim 1, wherein the dialysis membrane has a molecular weight cutoff of from 80 kDa to 100 kDa.

4. The device of claim 1, further comprising an implantable first guiding system configured for at least partially guiding the dialysis tube through a subcutaneous tissue.

5. The device of claim 1, further comprising a port providing access to the dialysis tubing from a body surface of the mammalian host and/or from a subcutaneous access element.

6. The device of claim 5, wherein the port is configured for transcutaneous implantation.

7. The device of claim 5, wherein the port comprises a fixing element configured to fix the port within or under the skin of the mammalian host.

8. The device of claim 1, wherein the dialysis tube is configured to be at least partially implanted intraperitoneally, subcutaneously, intraarterially, or intravenously.

9. The device of claim 1, wherein the device is configured for in situ replacement of the dialysis tube and/or for in situ replacement of the content of the dialysis tube.

10. The device of claim 1, wherein the therapeutic compound is selected from the group consisting of insulin, glucagon, Factor IX, human growth hormone (huGH), Calcitonin, Glucagon-like peptide (GLP-1) and agonists, Amylin, levodopa, somatostatin, alpha1-antitrypsin (AAT), interleukins, and interferons.

11. The device of claim 1, wherein the therapeutic compound is insulin or an insulin analog, wherein the plurality of cells is adapted to secrete an amount of insulin related to the concentration of glucose present in the matrix material.

12. A method of producing the device for implantation according to claim 1, comprising:
a) filling the dialysis tube with a plurality of cells secreting a therapeutic compound and a matrix material;
b) at least temporarily sealing at least one end of the dialysis tube; and
c) thereby producing the device for implantation.

13. A port system, comprising:
a device for implantation comprising a port according to claim 5; and
an extracorporeal connecting head.

14. A cell secreting a therapeutic compound for use in treating disease by implantation of said cells in a device for implantation according to claim 1.

15. A cell secreting a therapeutic compound for use in treating diabetes, Hemophilia B, huGH deficiency, osteoporosis, Parkinson's disease, acromegaly, an endocrine tumor, alpha1-antitrypsin-deficiency, an infectious disease, an autoimmune disease, cancer, or chronic virus infection by implantation of said cells in a device for implantation according to claim 1.
